SEQUENCE LISTING (1 sequence)

SEQ ID NO: 1 - DNA, 24 bp, Artificial Sequence
  Synthetic sequence of 24 nucleotides related to four immunostimulatory heximers of bacterial DNA
gacgtcgacgttaacgtcaacgtt

(19) United States
(12) Patent Application Publication
(10) Pub. No.: US 2004/0086557 A1
(43) Pub. Date: May 6, 2004

(54) Macrimmune V, a composition for treating cancer

(76) Inventor: James S. Lipton, West Palm Beach, FL (US)

Correspondence Address: S. Michael Bender, P.O. Box 530399, St. Petersburg, FL, 33747, US

(21) Appl. No.: 09/983,129

(22) Filed: Oct. 23, 2001

Related U.S. Application Data
(60) Provisional application No. 60/242,085, filed on Oct. 23, 2000.

(51) Int. Cl. A61K 38/16; C07H 21/04; C07K 14/415; A61K 9/127; C12N 15/88

(52) U.S. Cl. 424/450; 514/8; 435/458; 536/23.6; 530/370; 530/396

(57) ABSTRACT

The present invention is a composition for treating cancer composed of a novel oligonucleotide enclosed in a liposome together with Pokeweed Mitogen (PWM), which has been treated with activated polyethylene glycol PEG₂ to form PWM-PEG. The composition is given in conjunction with recombinant human interleukin-2, (rIL-2).

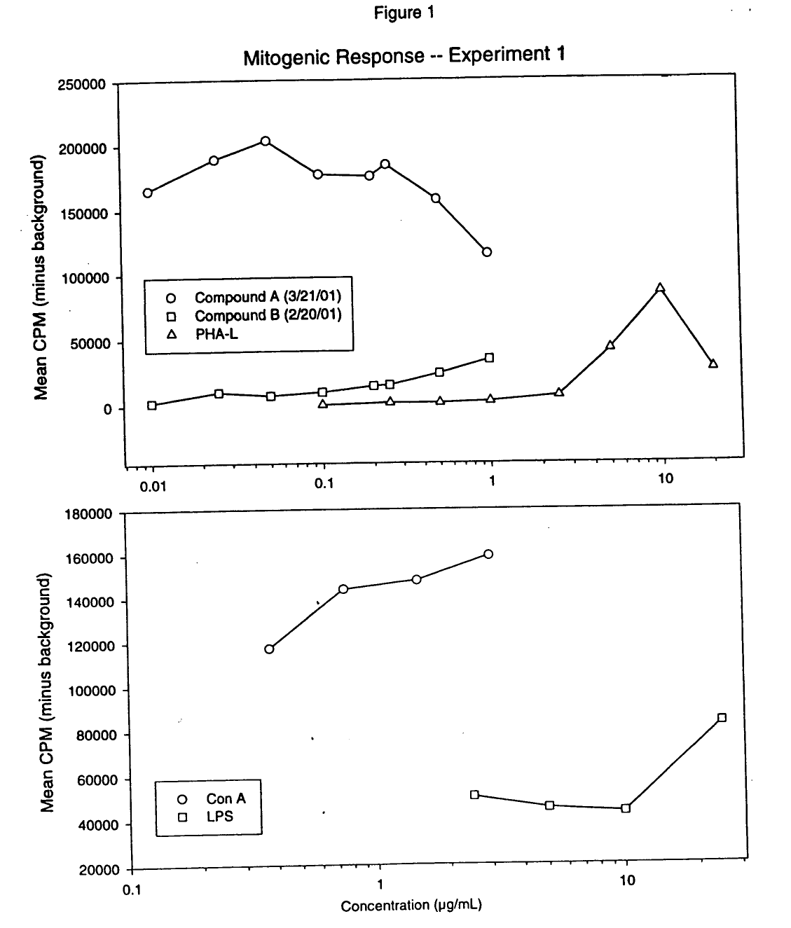

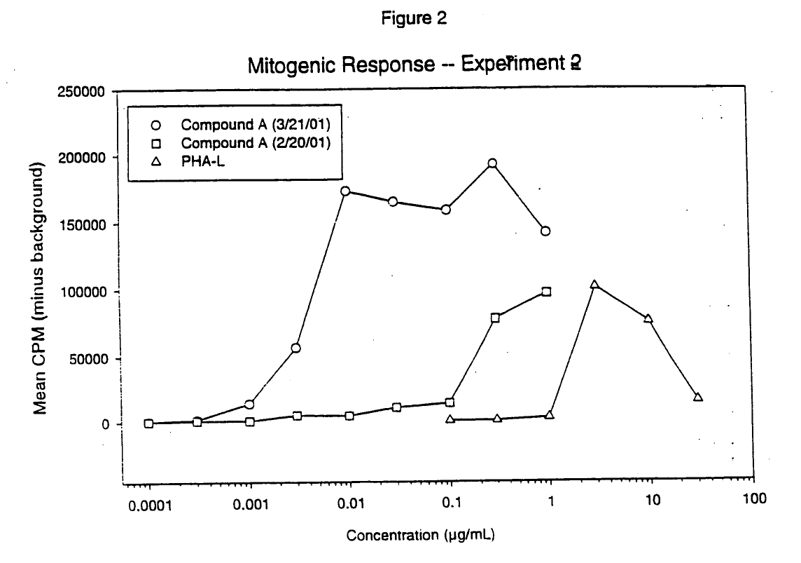

### DETAILED DESCRIPTION OF INVENTION

I am seeking a patent and trademark for an injectable drug called Macrimmune V. This drug is prepared as a mixture of two elements, Mitogen A:

Polyethylene glycol coated Pokeweed Mitogen (PWM-PEG) 0.05 μg PWM protein/kg

Recombinant Interleukin II (rIL-2) 100,00U./kg

The Pokeweed mitogen is prepared by the method of Borjeson¹. The root of the Pokeweed plant (phytolacca americana) is blenderized in a standard food processor/blender. The blenderized material is then extracted in PBS (phosphate buffered saline—0.01 M). The extraction is performed overnight @ 4 C., pH 7.3, using 1L of PBS per 1 lb. of pokeweed root. The supernatant is then filtered, the filtrate centrifuged @ 27,000 g for 15 min., and the precipitate removed. ¹Borjeson J. Reisfield R. and Chessin L. N. (1966) Journal of Experimental Medicine 124:859-872.

Now, TCA (trichloracetic acid 10%) is added to the solution. TCA is added volume for volume i.e. 1:1 at 0 C. The mixture is again centrifuged @ 27,000 g for 15 min. and applied to a hydroxylapatite column. The chromatographic peak is determined by O.D. (optical density) measure and the peak is eluted off in 0.005M phosphate @ pH 7.5.

Purified Pokeweed mitogen, in its final form, is composed of 5 proteins: Pₐ₁₋ₐ₅. Purified PWM, as a lyophilized powder, is available from several biochemical companies such as GIBCO/BRL in the U.S.A. and Seikagaku Kogyo Co. in Japan.

The PWM is complexed with the activated PEG2 ester PEG2-NHS(1-3), where NHS is N-hydroxysuccinimide. The protocol for the synthesis of PWM-PEG is as follows:

To 0.4 mg of PWM (2.5 mg/ml) dissolved in 0.5 M borate buffer (pH 10.0), 50 mg of activated PEG2 is added. The PEG2 is added slowly over a ten minute period @ 37 C. with constant stirring. The mixture is allowed to complete reaction over 1 h @ 37 C. with continued stirring. The resulting PWM-PEG is purified by dialysis against PBS, and ultrafiltration/concentration in an Amicon system with a PM 10 membrane (cut-off 10 kd) to eliminate N-hydroxysuccinimide and reduce the PEG concentration. The PWM-PEG is further purified from the unreacted PEG by gel filtration on a Pharmacia Superose 12 column, operated by an FPLC instrument, using 10 mM. phosphate buffer of pH 7.2, with 0.15M NaCl as eluent.

The activated PEG2-NHS can be obtained from Shearwater Polymers Inc., Huntsville, Ala. The above synthesis will put PEG on 52% of the free lysine molecules of 1 mg of PWM.

Recombinant human interleukin (rIL-2) is produced in E. Coli transfected with the gene from the Jurkit cell line². The protein is also available as a lyophilized powder from Chiron Pharmaceuticals (Proleuken). B ²Rosenberg S A, Grimm E A, McGrogan M, et al. Biological activity of recombinant human interleukin-2 produced in Escherichia coli. Science 1984; 4:1380-91.

The use of interleukin 2 as immunotherapy for advanced cancer is well established³. It is believed that the principal effect of IL-2is the activation of a subset of Natural Killer (NK) lymphocytes, thereby generating lymphokine activated killer cells (LAK). ³Rosenberg S A, Grimm E A, Muul L M, et al. A progress report on the treatment of 157 patients with advanced cancer using lymphokine-activated killer cells and interleukin-2 or high-dose interleukin-2 alone. N Eng J Med 1987; 316:889-97.

Subsequently, it was found that LAK cells could be further stimulated by certain plant lectins. This generates lectin dependant cell-mediated cytotoxicity, termed LDCC. The most potent lectin stimulation of human T and B-lymphocytes can be obtained with the use of pokeweed mitogen, (PWM).

The problem with PWM is that it is highly immunogenic. The Japanese have solved the problem of pokeweed's immunogenicity by conjugating it with activated polyethylene glycol (PEG)⁴. The resulting PWM-PEG conjugate is not as immunogenic as the pure PWM but it is, also, less active. The use of the NHS ester to activate the PEG rather than the 6-chloro-s-triazine used by the Japanese has improved the mitogenic activity of the PWM-PEG. ⁴Ueno T et al. (1996) Journal of Biomaterial Science, Polymer Edition 7(9):753-8.

The second element of the invention is Mitogen B:

This is the oligonucleotide which is illustrated in SEQ No I.

The immunostimulatory activity of bacterial DNA is well known. Recently the exact nature of this stimulatory motif has been determined. A CG dinucleotide is essential, flanked by two, 5′ purines and two, 5′ pyrimidines. The four most stimulating heximers have been found to be GACGTC, GACGTT, AACGTC, and AACGTT.⁷ ⁷Krieg, A M 1996. Lymphocyte activation by CpG dinucleotide motifs in prokaryotic DNA Trends Microbiol, 4:73

Immunostimulatory activity of cationic lipid-DNA complexes have been noted when bacterial DNA was employed (in the form of plasmids).⁸ We have noted that liposomal-CG-oligonucleotides also cause cytokine and cellular stimulation. This also appears to be the case when Poly(I:C) is used as the DNA source. ⁸Freimark, B D, et al. 1998. Cationic lipids enhance cytokine and cell influx levels in the lung following administration of plasmid:cationic lipid complexes. J. Immunol. 160:4580

We have prepared an oligonucleotide from the four most stimulating hexamers. DNA synthesis involves protection of the 5′ end of the first nucleotide by dimethoxytrityl(DMT) while the OH end is attached by a linker to silica. Afterwards DMT is removed by washing and the next nucleotide is activated and attached. Using iodine, 5′, 3′ linkage is oxidized to generate a phosphotriester bond, and one of the phosphate oxygen's is methylated. The reaction has been automated to 80 groups.⁹ ⁹Ellington, A and Green, R 1990. Synthesis of Oligonucleotides. IN Current Protocols in Molecular Biology (F. M. Ausubel et al. eds.)pp.2.11.1-2.11.18, Green Publishing and Wiley-Interscience, New York.

After automated synthesis, the nucleotides are purified using polyacrylamide gel electrophoresis (PAGE). They are then incorporated in cationic lipids. Briefly: DOTAP(1, 2 dioleoyl-3-trimethylammonium-propane) Avanti Polar Lipids, Alabaster, Ala. and cholesterol(Sigma) are mixed in a 1:1 molar ratio, and dried in the bottom of round flasks. They are then rehydrated with 5% dextrose at 50 C. for 6 h. ¹⁰ ¹⁰Liu, Y D et al. 1995, Cationic liposome-mediated i.v. gene delivery in mice. J. Biol. Chem. 27:24864.

DNA is added at a ratio of 30 nmol lipid to 1 μgDNA to a final concentration of 100 μg DNA per 0.1 ml. dextrose. The dose of DNA necessary for maximal immunostimulation is about 50-100 μg/kg.

### EXPERIMENT 1

Eighty C57BL x DBA mice were divided into groups of 5. They were given either Meth A or EL-4 tumors at a dose of 5×10⁵ cell, injected s.c. in the suprascapular region. They were Meth then treated with either mitogen-A, mitogen-B, both or neither at 1 or at 3 days post tumor implant. Animals were 6-8 weeks old and weight matched to 20 g +/−3 g (SD). They were given mitogen-A at a dose of 50 μg i.p. and mitogen-B at 10 μg (0.1 ml soln) i.v. Mitogen-A was given biweekly for up to three weeks. Mitogen-B was given weekly for up to 3 weeks. Tumor volumes are estimated in mm³+/−SD.

TABLE 1
Effect of single agent or combination therapy given on day 1 post tumor implant

| Tumor | Agent | Day 7 | | Day 10 | | Day 14 | | Day 21 | | Cures | | Deaths | |
|---|---|---|---|---|---|---|---|---|---|---|---|---|---|
| MethA | Saline | | 0 | 153(22) | | 450(60) | | 5000(3500 | | | 0 | | 5 |
| MethA | Mit A | | 0 | | 0 | | 0 | | 0 | | 5 | | 0 |
| MethA | Mit B | | 0 | | 0 | | 0 | | 0 | | 5 | | 0 |
| MethA | Mit A & B | | 0 | | 0 | | 0 | | 0 | | 5 | | 0 |
| EL-4 | Saline | 75(25) | | 250(100) | | 500(150) | | TL | | | 0 | | 5 |
| EL-4 | Mit A | P4 | | | 0 | | 0 | | 0 | | 5 | | 0 |
| EL-4 | Mit B | | 0 | | 0 | | 0 | | 0 | | 5 | | 0 |
| EL-4 | Mit A & B | | 0 | | 0 | | 0 | | 0 | | 5 | | 0 |

TL = too large &/or death
P = palpable # animals

TABLE 2
Effect of single agent or combination therapy given on day 3 post tumor

| Tumor | Agent | day 7 | | day 10 | | day 14 | | day 21 | | Cures | | Deaths | |
|---|---|---|---|---|---|---|---|---|---|---|---|---|---|
| Meth A | Saline | P3 | 0 | 100(25) | | 500(50) | | 5500(3500 | | | 0 | | 5 |
| Meth A | Mit A | P5 | | | 0 | | 0 | | 0 | | 5 | | 0 |
| Meth A | Mit B | P5 | | P3 | | | 0 | | 0 | | 5 | | 0 |
| Meth A | Mit A & B | | 0 | | 0 | | 0 | | 0 | | 5 | | 0 |
| EL-4 | Saline | 80(20) | | 250(100) | | 750(175) | | LT | | | 0 | | 5 |
| EL-4 | Mit A | P5 | | P1 | | | 0 | | 0 | | 5 | | 0 |
| EL-4 | Mit B | P1 | | P1 | | | 100 | | 250 | | 4 | | 1 |
| EL-4 | Mit A & B | P4 | | P1 | | | 0 | | 0 | | 5 | | 0 |

P = palpable #
LT = tumor too large &/or death

### EXPERIMENT 2

Forty C57BL x DBA were given an intraperitoneal injection of 10⁵ cells from EL-4 or Meth A, followed by i.p. injections of Mitogens A, B or both on day 1 after tumor implantation. Dosages of mitogens is the same as in Experiment 1.

TABLE 3
Effect of single or combined agent therapy on i.p. tumors

| Tumor | Agent | # animals | | deaths | | days till death | | cures | |
|---|---|---|---|---|---|---|---|---|---|
| Meth A | Saline | | 5 | | 5 | 25-33(27) | | | 0 |
| Meth A | Mit A | | 5 | | 5 | 21-43(38) | | | |
| Meth A | Mit B | | 5 | | 5 | 29-45(42) | | | |
| Meth A | Mit A & B | | 5 | | 0 | | | | 5 |
| EL-4 | Saline | | 5 | | 5 | 7-9(8) | | | 0 |
| EL-4 | Mit A | | 5 | | 5 | 20-47(40) | | | 0 |
| EL-4 | Mit B | | 5 | | 3 | 40-47(43) | | | 2 |
| EL-4 | Mit A & B | | 5 | | 1 | | 57 | | 4 |

### Mitogenic Properties of Compounds A and B

### Test Articles and Reagents

A and B were prepared as indicated. Concavalin A (Con A), lipopolysaccharide (LPS), and PHA-L were purchased from Sigma Chemical Co. (St. Louis, Mo.). All diluted samples and controls were filtered through a 0.2 μm filter to sterilize the stock solutions prior to assay.

### Cells

Female C57BL/6 mice were obtained from Charles River Laboratories (Raleigh, N.C.). Mice were approximately 6-8 weeks of age when used. Mice were sacrificed by CO₂ inhalation and spleens were removed aseptically. Single cell suspensions were prepared by disaggregating the cells with frosted glass slides. Cells were washed twice and resuspended in complete medium. Complete medium sonsisted of RPMI-1640 medium containing 25 mM HEPES buffer (Mediatech, Herndon Va.) supplemented with 10% fetal bovine serum, 100 μg/ml streptomycin, 100 μg/ml penicillin, 10 μml gentamicin (GIBCO-BRL, Gaithersburg, Md.), 2 mM L-glutamine (Mediatech), and 2×10⁻⁵ M 2-mercaptoethanol (Sigma).

### Proliferation Assays

Spleen cells (2×10⁵/well) were placed in 96-well flat micrometer plates Costar/Corning, Corning, N.Y.) and cultured in triplicate with either medium (no stimulus or background) or various concentrations of PHA-L, Con A, LPS, and compounds A and B. Cultures were incubated at 37° C. in humidified 5% CO₂ for three days, pulsed with 1 μCi³H-thymidine (³H-TdR)/well for the final 6-16 hours of incubation, and harvested using a Skatron (Sterling, Va.) semi-automated harvester. Proliferation was measured by ³H-TdR incorporation after counting samples in a Beckman LS 60001C (Fullerton, Calif.) liquid scintillation counter. Data were processed using Microsoft Excel software. The first experiment compares Mitogens/Compounds A and B with PHA-L.

TABLE 1
Experiment 1 - Compound A (Mar. 21, 2001) and Compound B (Feb. 20, 2001)

| | | Proliferative Response | | | | Proliferative Response (minus Background) | | | |
|---|---|---|---|---|---|---|---|---|---|
| Agent | Dosage | Raw CPM | | | Mean CPM ± SD | Raw CPM | | | Mean CPM ± SD |
| Media | — | 1654 | 1765 | 1572 | 1735 ± 294 | | | | |
| | | 1305 | 2101 | 2015 | | | | | |
| | | 2236 | 2063 | 3008 | 2407 ± 556 | 501 | 328 | 1273 | 671 ± 556 |
| | | 3207 | 1998 | 1927 | | 1472 | 263 | 192 | |
| Compound A | 1.000 μg/mL | 109961 | 128564 | 110816 | 116447 ± 10502 | 108226 | 126829 | 109081 | 114712 ± 10502 |
| (Mar. 21, | 0.500 μg/mL | 156931 | 163253 | 157019 | 159068 ± 3625 | 155196 | 161518 | 155284 | 157333 ± 3625 |
| 2001) | 0.250 μg/mL | 195449 | 176014 | * | 185732 ± 13743 | 193714 | 174279 | * | 183996 ± 13743 |
| | 0.200 μg/mL | 179208 | 175642 | 176595 | 177149 ± 1846 | 177473 | 173907 | 174860 | 175413 ± 1846 |
| | 0.100 μg/mL | 177943 | 166293 | 191647 | 178627 ± 12691 | 176207 | 164557 | 189911 | 176892 ± 12691 |
| | 0.050 μg/mL | 209626 | 173578 | 232072 | 205092 ± 29510 | 207890 | 171842 | 230338 | 203356 ± 29510 |
| | 0.025 μg/mL | 203103 | 191088 | 177898 | 190696 ± 12607 | 201367 | 189352 | 176162 | 188961 ± 12607 |
| | 0.010 μg/mL | 159906 | 153294 | 187558 | 166919 ± 18177 | 158170 | 151558 | 185822 | 165184 ± 18177 |
| Compound B | 1.000 μg/mL | 35639 | 35395 | 36498 | 35844 ± 579 | 33904 | 33660 | 34763 | 34109 ± 579 |
| (Feb. 20, 2001) | 0.500 μg/mL | 22485 | 29819 | 23886 | 25397 ± 3893 | 20750 | 28084 | 22151 | 23661 ± 3893 |
| | 0.250 μg/mL | 17321 | 17373 | 14986 | 16560 ± 1363 | 15586 | 15838 | 13251 | 14825 ± 1363 |
| | 0.200 μg/mL | 15263 | 16211 | 18462 | 15979 ± 632 | 13528 | 14476 | 14727 | 14243 ± 632 |
| | 0.100 μg/mL | 11496 | 13790 | 9560 | 11615 ± 2118 | 9761 | 12055 | 7825 | 9880 ± 2118 |
| | 0.050 μg/mL | 9258 | 7905 | 9959 | 9041 ± 1044 | 7523 | 6170 | 8224 | 7305 ± 1044 |
| | 0.025 μg/mL | 8028 | 7520 | 19522 | 11690 ± 6788 | 6293 | 5785 | 17787 | 9955 ± 6788 |
| | 0.010 μg/mL | 4122 | 4311 | 3534 | 3989 ± 405 | 2387 | 2576 | 1799 | 2254 ± 405 |
| PHA-L | 20.00 μg/mL | 30457 | 31747 | 22272 | 28159 ± 5139 | 28722 | 30012 | 20537 | 28424 ± 5139 |
| | 10.00 μg/mL | 81240 | 83980 | 95301 | 86840 ± 7454 | 79504 | 82244 | 93565 | 85105 ± 7454 |
| | 5.00 μg/mL | 42263 | 54563 | 33665 | 43497 ± 10504 | 40528 | 52828 | 31930 | 41762 ± 10504 |
| | 2.50 μg/mL | 7492 | 7648 | 9452 | 8197 ± 1089 | 5757 | 5913 | 7717 | 6462 ± 108 |
| | 1.00 μg/mL | 5408 | 2417 | 4238 | 4021 ± 1507 | 3673 | 682 | 2503 | 2286 ± 150 |
| | 0.50 μg/mL | 2876 | 2238 | 3844 | 2986 ± 809 | 1141 | 503 | 2109 | 1251 ± 809 |
| | 0.25 μg/mL | 2198 | 3859 | 3306 | 3121 ± 848 | 483 | 2124 | 1571 | 1386 ± 846 |
| | 0.10 μg/mL | 2045 | 1880 | 1902 | 1942 ± 90 | 310 | 145 | 167 | 207 ± 90 |
| Con A | 3.000 μg/mL | 156233 | 151182 | 174547 | 160654 ± 12294 | 154497 | 149446 | 172812 | 158919 ± 12294 |
| | 1.500 μg/mL | 147906 | 151367 | * | 149636 ± 2447 | 148170 | 149631 | * | 147901 ± 2447 |
| | 0.750 μg/mL | 150280 | 159314 | 127738 | 145777 ± 16263 | 148544 | 157579 | 126002 | 144042 ± 16263 |
| | 0.375 μg/mL | 130588 | 103471 | 122789 | 118949 ± 13960 | 128852 | 101736 | 121053 | 117214 ± 13960 |
| LPS | 25.0 μg/mL | 88172 | 81155 | * | 84664 ± 4962 | 86437 | 79420 | * | 82928 ± 4962 |
| | 10.0 μg/mL | 46551 | 48269 | 39792 | 44870 ± 4481 | 44815 | 46533 | 38056 | 43135 ± 4481 |
| | 5.0 μg/mL | 46215 | 50369 | 43859 | 46814 ± 3296 | 44479 | 48633 | 42123 | 45079 ± 3296 |
| | 2.5 μg/mL | 40418 | 60794 | 54771 | 51994 ± 10468 | 38682 | 59059 | 53036 | 50259 ± 10468 |

*Outlier deleted.

1. A composition (Mitogen A) that includes a plant mitogenic lectin (PWM), that is combined with an immunogenicity modifier(PEG and recombinant Human Interleukin II), so that the lectin does not cause an undesired reaction between T and B cells in the acquired immune system when administered systemically.

2. The oligonucleotide comprising sequence No. 1

3. A composition (Mitogen B) comprising the oligonucleotide of claim 2, enclosed in a liposome.

4. A composition (Macrimmune V) comprising Mitogen B of claim 3 and Mitogen A, of claim 1.

5. The composition of claim 4 in which where the oligonucleotide is replaced by an equal weight of Poly(I:C).

6. The composition of claim 1, in which the PWM has been replaced by the isolectin Lsub4 of phytohemagglutinin (PHA-Lsub4).
